(19) United States
(12) Patent Application Publication
(10) Pub. No.: US 2006/0216257 A1
(43) Pub. Date: Sep. 28, 2006

(54) Makeup and/or care kit providing volumizing effect

(75) Inventors: Karl Pays, Saint-Maurice (FR); Yohann Bichon, Maisons-Alfort (FR)

Correspondence Address: OLIFF & BERRIDGE, PLC, P.O. BOX 19928, ALEXANDRIA, VA, 22320, US

(73) Assignee: L'OREAL, Paris (FR)

(21) Appl. No.: 11/377,587

(22) Filed: Mar. 17, 2006

Related U.S. Application Data
(60) Provisional application No. 60/670,288, filed on Apr. 12, 2005.

(30) Foreign Application Priority Data
Mar. 24, 2005 (FR) 05 50774

(51) Int. Cl. A61K 8/73; A61K 8/81

(52) U.S. Cl. 424/70.7; 424/70.16; 424/70.13

(57) ABSTRACT

The present invention relates to a kit for making up and/or caring for the skin, the lips and keratin fibres, comprising at least: one cosmetic composition with an oily continuous phase comprising at least one polyelectrolyte, and one aqueous composition.

The present invention relates to a makeup kit, especially for the skin, the lips and/or keratin fibres, comprising at least one aqueous composition and at least one cosmetic composition with an oily continuous phase, which can give a volumizing effect when the said compositions are placed in contact.

More particularly, the makeup kit according to the invention may constitute a makeup product for the face, the body and/or the lips.

The present invention also relates to the making up of keratin fibres, for instance the eyelashes, the eyebrows and the hair, and more particularly to making up the eyelashes.

The makeup kit according to the invention may be in the form of a makeup kit for the eyelashes or the eyebrows, or a hair makeup kit. More especially, the invention relates to a mascara. It may especially be a makeup kit or a treatment kit for the eyelashes.

In general, compositions for making up keratin fibres and especially the eyelashes comprise a liquid fatty phase (oils) containing an oily structuring agent, which may be a wax, a polymer, in particular a semi-crystalline polymer, or a lipophilic gelling agent.

There are in practice essentially two types of mascara formulation, i.e. firstly, mascaras with an aqueous continuous phase, known as “emulsion mascaras”, which are in the form of a wax-in-water emulsion, and, secondly, mascaras with a solvent or oil continuous phase, which are anhydrous or have a low content of water and/or of water-soluble solvents, known as “waterproof mascaras”, which are formulated in the form of a dispersion of waxes in non-aqueous solvents.

The term “composition with an aqueous continuous phase” means that the composition has a conductivity, measured at 25° C., of greater than or equal to 23 μS/cm (microSiemens/cm), the conductivity being measured, for example, using an MPC227 conductimeter from Mettler Toledo and an Inlab 730 conductivity measuring cell. The measuring cell is immersed in the composition, so as to remove the air bubbles liable to form between the two electrodes of the cell. The conductivity reading is taken when the conductimeter value has stabilized. A mean is calculated on at least three successive measurements.

The term “composition with a solvent continuous phase” means that the composition has a conductivity, measured at 25° C., of less than 23 μS/cm (microSiemens/cm), the conductivity being measured as indicated above.

The term “give volume”, also termed below a volumizing effect, is an effect frequently sought in the cosmetics field.

In the field of eyelash makeup, very concentrated mascara formulations with a high solids content have been proposed, for example, making it possible to deposit a large thickness of material. The principle of these thickening mascaras is, in other words, to deposit the maximum amount of material on the eyelashes. However, such formulations have the drawback of having a very thick consistency and thus of being difficult to apply. The major drawback is the formation of “blobs”, which are considered unattractive. Still in the field of mascaras, very fluid formulations have, conversely, been proposed, which, by superposition of coats, also allow a volumizing effect to be obtained. However, in this case also, the conditions for obtaining the volumizing effect are not entirely satisfactory since they are long and difficult to implement.

For the lips, the volumizing effect has mainly been obtained hitherto either by drawing a lip contour that is larger than the natural contour, or by playing with optical effects, for example using glossy compositions.

Another alternative consisted in using, in cosmetic compositions, polymers with a high water-absorbing capacity. Placing the composition, applied to its area of makeup, in contact with water causes an increase in its initial volume, thus generating the desired volumizing effect. Such compositions are especially described in documents U.S. Pat. No. 6,045,783 and EP 1 228 747. However, the compositions with a volumizing effect using these polymers are relatively unsatisfactory since quite a large amount of water is needed to obtain significant swelling.

Documents FR 2 785 801, EP 1 152 022, FR 2 774 996, WO 95/35089 and WO 99/36445 describe thickening compositions referred to as “thickening latices”, “thickeners” or “inverse latices”.

Patent applications FR 2 785 801 and FR 2 774 996 especially disclose compositions comprising an aqueous phase, an oily phase, an emulsifier of O/W (oil-in-water) type and an emulsifier of W/O (water-in-oil) type, and also a branched or crosslinked anionic polyelectrolyte based on a monomer containing a strong acid function.

Document WO 99/52499 mainly describes lipsticks comprising a sodium polyacrylate with the aim of producing a volumizing effect.

It has now been discovered that it is possible to obtain makeup kits capable of generating a particularly advantageous volumizing effect while at the same time maintaining good separation of the eyelashes.

According to a first aspect, the present invention relates to a kit for making up and/or caring for the skin, the lips and/or keratin fibres, comprising at least:

one cosmetic composition with an oily continuous phase comprising at least one polyelectrolyte, and

one aqueous composition.

The inventors have thus observed that by placing an aqueous composition in contact with a cosmetic composition in accordance with the present invention, irrespective of the order in which these two compositions are deposited, a particularly advantageous volumizing effect in terms of makeup can be generated.

According to a second aspect, the present invention relates to a non-therapeutic cosmetic process for making up and/or caring for the skin, the lips and/or keratin fibres, comprising at least one step of applying a cosmetic composition as defined above to the skin, the lips and/or the keratin fibres, and at least one step of applying an aqueous composition.

According to a third aspect, a subject of the present invention is a made-up support comprising a makeup that may be obtained according to the process as defined above.

According to a fourth aspect, the present invention relates to the use of at least one polyelectrolyte for the preparation of a kit for making up the skin, the lips and/or keratin fibres, comprising at least one cosmetic composition with an oily continuous phase and at least one aqueous composition, as additive for the said cosmetic composition with an oily continuous phase, to obtain a volumizing effect when the said compositions are placed in contact.

As emerges from the examples hereinbelow, the makeup kits according to the invention show such technical advantages.

In the context of the present invention, the term “keratin fibres” especially means the hair, the eyelashes and the eyebrows. Furthermore, the term “making up the skin” especially means making up the body, the hands, the neck or the face.

The makeup kits in accordance with the invention comprise a physiologically acceptable medium, especially a cosmetically acceptable medium, i.e. a medium that is compatible in particular with keratin fibres such as the hair, the eyelashes and the eyebrows.

In the context of the present invention, the term “cosmetically acceptable” means a compound whose use is compatible with application to keratin materials.

For the sake of simplicity, unless otherwise mentioned, the term “cosmetic composition with an oily continuous phase” or “cosmetic composition” or “cosmetic composition in accordance with the invention” or “cosmetic composition included in the kit” denotes the cosmetic composition with an oily continuous phase comprising at least one polyelectrolyte, as included in the makeup kit according to the present invention.

Thus, the user can obtain the desired volumizing effect by means of at least two application actions.

In the context of the present invention, the terms “volumizing effect”, “swelling effect” and “thickening effect”, which qualify the makeup resulting from the application of the two compositions included in the claimed kit, are used without discrimination. It is noted that the term “swelling effect” is particularly suitable for making up and/or caring for keratin fibres.

The volumizing effect may advantageously become apparent via a swelling effect of the eyelashes. This swelling effect is all the more apparent when the cosmetic composition in accordance with the invention is deposited first and the aqueous composition is deposited second, and when the cosmetic composition in accordance with the invention contains dyestuffs and the aqueous composition is advantageously transparent or translucent. In this case of making up the eyelashes, swelling of the coat on the eyelashes may thus be seen with the naked eye, as soon as the aqueous composition has been deposited. In other words, the cross section of the coated eyelash, after applying the aqueous composition, increases compared with the cross section of the eyelash coated only by applying the cosmetic composition in accordance with the present invention.

However, this embodiment of the invention has no limiting nature, and as such there is no departure from the context of the present invention especially when the order of application of the compositions is different.

Depending on the order of application, one or other of the cosmetic composition or the aqueous composition may be termed “base coat” or “top coat”.

Polyelectrolyte

The term “polyelectrolyte” means a macromolecular substance that has the capacity of dissociating when it is dissolved in water or in any other ionizing medium, to give at least one ion. In other words, a polyelectrolyte is a polymer comprising at least one ionizable monomer.

In particular, the polyelectrolyte may give polyions, for example polyanions, when it is dissociated in water. A polyelectrolyte may be a polyacid, a polybase, a polysalt or a polyampholyte. In the context of the invention, it is preferably a polyacid, and preferably a strong polyacid.

Preferably, the polyelectrolyte included in the cosmetic compositions according to the present invention is a branched and/or crosslinked anionic polymer.

Preferably, the polyelectrolyte is also capable of forming a gel in aqueous solution, above a concentration of greater than or equal to 0.1% by weight and preferably ≧0.3% by weight of solids relative to the total weight of the composition. This gel may be characterized, by oscillatory rheology (v=1 Hz), by a flow threshold τ_{c} at least equal to 10 Pa.

Furthermore, when the cosmetic composition according to the present invention comprises a film-forming polymer, the polyelectrolyte is advantageously different from this film-forming polymer.

The counterions of the polyions formed during the dissociation may be mineral or organic, of any nature.

In particular, when the polyelectrolyte is a branched or crosslinked anionic polymer, the cations may be alkali metal or alkaline-earth metal cations such as sodium or potassium, or alternatively the ammonium ion.

The sodium cation Na⁺ is preferred, which is why it is mainly cited in the list of polyelectrolytes that follows, without this constituting any limitation to this specific counterion.

Polyelectrolytes that may be mentioned include:

the acrylamide/Na AMPS copolymer such as Simulgel 600® in emulsion form containing polysorbate 80 as surfactant and containing isohexadecane as oil phase, sold by the company SEPPIC, or alternatively Simulgel EG®, Simulgel A® and Simulgel 501® sold by the same company.

Simulgel 600® is especially described in document FR 2 785 801. It is in effect an inverse latex. The AMPS polyelectrolyte is 2-methyl-2-[(1-oxo-2-propenyl)amino]-1-propanesulfonic acid partially or totally salified especially in sodium salt or ammonium salt form to a proportion of 30 mol % to 50 mol % in the mixture comprising AMPS and also an acrylamide, which is itself present in a proportion of 50% to 70%.

crosslinked sodium starch glycolate in powder form,

sodium polyacrylates such as Norsocryl S35® sold by the company Atofina, or Cosmedia SP® sold by the company Cognis,

ionizable polysaccharide derivatives such as cellulose salts and sodium alginates,

starch-based grafted copolymers such as the Waterlock® products (for example A-180 and G-400) from Grain Processing Corporation,

polyacrylic acids of Synthalen K® type,

polyacrylic acid alkyl acrylate copolymers of Pemulen® type,

AMPS (polyacrylamidomethylpropanesulfonic acid partially neutralized with ammonia and highly crosslinked) sold by the company Clariant,

polyoxyethylenated AMPS/alkyl methacrylate copolymers (crosslinked or non-crosslinked),

sodium carboxymethylcellulose and any ionizable cellulose derivative, and

mixtures thereof.

Sodium polyacrylate and acrylamide/AMPS copolymer and copolymers thereof are most particularly suitable for use in the invention.

It will obviously be arranged for the polyelectrolyte content to be adjusted such that the capacity to give a volumizing effect, and/or a swelling effect when it is a matter of making up keratin fibres, is effectively exhibited.

It is understood that the amount of polyelectrolyte can vary significantly depending on the nature of the polyelectrolyte. In general, this amount is at least equal to the amount that is necessary and sufficient to give the said composition a volumizing effect. It is also termed the effective amount.

According to one preferred variant, the polyelectrolyte is present in the cosmetic composition with an oily continuous phase preferably in a solids content ranging from 0.05% to 40% by weight, more preferably from 1% to 20% by weight and better still from 5% to 10% by weight relative to the total weight of the composition.

Without this constituting any limitation to the invention, the inventors have put forward the theory that the polyelectrolyte dispersed in the oil acts as a “water pump”. Thus, this “water pump” role is more clearly seen when the composition is placed in contact with an aqueous phase. Due to the presence of the high osmotic pressure in the aqueous reservoirs containing the polyelectrolyte, on account of the presence of counterions, the said reservoirs swell.

Thus, at the macroscopic level, once the two compositions have been deposited, i.e. placed in contact, the film of the cosmetic composition in accordance with the invention becomes hydrated by means of the numerous “water pumps” microdispersed in the film. The said film swells and the volumizing effect, and/or the swelling effect when it is makeup for keratin fibres, becomes apparent.

Oils

The cosmetic composition included in the makeup kit according to the invention may comprise one or more oils or organic solvent.

The said cosmetic composition with an oily continuous phase usually comprises a total amount of oil ranging from 10% to 90%, preferably from 15% to 80% and better still from 20% to 60% by weight relative to the total weight of the composition.

The oil(s) present in the composition of the invention may be chosen from volatile oils and/or non-volatile oils, and mixtures thereof.

Cosmetic compositions essentially comprising volatile oils are preferred in the context of the present invention.

The term “volatile organic solvent or oil” means an organic solvent or oil (or non-aqueous medium) capable of evaporating on contact with the skin in less than one hour, at room temperature and atmospheric pressure.

The volatile oil is a volatile cosmetic oil, which is liquid at room temperature, especially having a non-zero vapour pressure, at room temperature and atmospheric pressure, in particular having a vapour pressure ranging from 0.13 Pa to 40 000 Pa (10⁻³ to 300 mmHg) and preferably ranging from 1.3 Pa to 8000 Pa (0.01 to 60 mmHg).

The volatile oils (or organic solvents) may be hydrocarbon-based oils, silicone oils or fluoro oils, or mixtures thereof

The term “hydrocarbon-based oil” means an oil mainly containing hydrogen and carbon atoms and possibly oxygen, nitrogen, sulfur and/or phosphorus atoms. The volatile hydrocarbon-based oils may be chosen from hydrocarbon-based oils containing from 8 to 16 carbon atoms, and especially branched C₈-C₁₆ alkanes, for instance C₈-C₁₆ isoalkanes of petroleum origin (also known as isoparaffins), for instance isododecane (also known as 2,2,4,4,6-pentamethylheptane), isodecane, isohexadecane, and for example the oils sold under the trade names “Isopar®” or “Permethyl®”, branched C₈-C₁₆ esters, isohexyl neopentanoate, and mixtures thereof. Other volatile hydrocarbon-based oils, for instance petroleum distillates, especially those sold under the name “Shell Solt®” by the company Shell, may also be used.

Volatile oils that may also be used include volatile silicones, for instance volatile linear or cyclic silicone oils, especially those with a viscosity ≦6 centistokes (6×10⁻⁶ m²/s) and especially containing from 3 to 6 silicon atoms, these silicones optionally comprising one or more alkyl or alkoxy groups containing 1 or 2 carbon atoms. As volatile silicone oils that may be used in the invention, mention may be made especially of octamethylcyclotetrasiloxane, decamethylcyclopentasiloxane, dodecamethylcyclo-hexasiloxane, heptamethylhexyltrisiloxane, heptamethyloctyltrisiloxane, hexamethyl-disiloxane, octamethyltrisiloxane, decamethyltetrasiloxane, dodecamethylpentasiloxane, heptamethylethyltrisiloxane and heptamethylbutyltrisiloxane, and mixtures thereof

Volatile organic solvents, especially fluorinated solvents such as nonafluoro-methoxybutane or perfluoromethylcyclopentane, may also be used.

Advantageously, the volatile oil(s) is or are chosen from hydrocarbon-based volatile oils containing from 8 to 16 carbon atoms, such as isododecane, volatile silicone oils such as decamethylcyclopentasiloxane (D5) or dodecamethylcyclohexasiloxane (D6), and mixtures thereof.

The compositions according to the invention may also comprise at least one non-volatile compound, which is water-insoluble and liquid at room temperature, especially at least one non-volatile organic solvent or oil, which may be chosen in particular from non-volatile hydrocarbon-based oils and/or silicone oils and/or fluoro oils.

Non-volatile hydrocarbon-based oils that may especially be mentioned include:

hydrocarbon-based oils of plant origin, such as triglycerides consisting of fatty acid esters of glycerol, the fatty acids of which may have varied chain lengths from C₄ to C₂₄, these chains possibly being linear or branched, and saturated or unsaturated; these oils are especially wheatgerm oil, sunflower oil, grapeseed oil, sesame seed oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppyseed oil, pumpkin oil, sesame seed oil, marrow oil, rapeseed oil, blackcurrant oil, evening primrose oil, millet oil, barley oil, quinoa oil, rye oil, safflower oil, candlenut oil, passionflower oil or musk rose oil; or caprylic/capric acid triglycerides, for instance those sold by the company Stéarineries Dubois or those sold under the names Miglyol 810®, 812® and 818® by the company Dynamit Nobel,

synthetic ethers containing from 10 to 40 carbon atoms,

linear or branched hydrocarbons of mineral or synthetic origin, such as petroleum jelly, polydecenes, hydrogenated polyisobutene such as parleam, and squalane, and mixtures thereof,

synthetic esters, for instance oils of formula R₁COOR₂ in which R₁ represents a linear or branched fatty acid residue containing from 1 to 40 carbon atoms and R₂ represents a hydrocarbon-based chain, which is especially branched, containing from 1 to 40 carbon atoms, on condition that R₁+R₂≧10, for instance purcellin oil (cetostearyl octanoate), isopropyl myristate, isopropyl palmitate, C₁₂ to C₁₅ alkyl benzoate, hexyl laurate, diisopropyl adipate, isononyl isononanoate, 2-ethylhexyl palmitate, isostearyl isostearate, alcohol or polyalcohol octanoates, decanoates or ricinoleates, for instance propylene glycol dioctanoate; hydroxylated esters, for instance isostearyl lactate or diisostearyl malate; and pentaerythritol esters,

fatty alcohols that are liquid at room temperature with a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol,

higher fatty acids such as oleic acid, linoleic acid or linolenic acid, and mixtures thereof.

The non-volatile silicone oils that may be used in the composition according to the invention may be non-volatile polydimethylsiloxanes (PDMS), polydimethylsiloxanes comprising alkyl or alkoxy groups, which are pendent and/or at the end of a silicone chain, these groups each containing from 2 to 24 carbon atoms, phenyl silicones, for instance phenyl trimethicones, phenyl dimethicones, phenyltrimethylsiloxydiphenylsiloxanes, diphenyl dimethicones, diphenyhnethyldiphenyltrisiloxanes and 2-phenylethyltrimethyl-siloxysilicates.

The fluoro oils that may be used in the composition of the invention are especially fluorosilicone oils, fluoro polyethers or fluoro silicones as described in document EP-A-847 752.

The content of volatile organic solvent or oil in the cosmetic composition in accordance with the invention may range from 20% to 80% by weight, in particular from 30% to 70% by weight and better still from 35% to 60% by weight relative to the total weight of the composition.

The content of non-volatile organic solvent or oil in the cosmetic composition in accordance with the invention may range from 0.01% to 30% by weight, in particular from 0.1% to 25% by weight and better still from 0.1% to 20% by weight relative to the total weight of the composition.

Aqueous Composition

For the purposes of the invention, the term “aqueous composition” denotes a composition comprising an amount of water and/or of water-soluble solvent as defined below that is sufficient to generate, when placed in contact with the cosmetic composition in accordance with the invention, swelling of this composition most probably due to the osmotic phenomenon mentioned above.

As aqueous composition, any aqueous composition known to those skilled in the art may be used.

It may contain one or more polymer(s) capable of forming a hydrophobic film.

In this respect, reference may be made to the hydrophobic polymers as defined below.

According to one preferred embodiment, the aqueous composition contains at least 15% by weight, preferably at least 20% by weight and even more preferably at least 25% by weight of a film-forming polymer as described below, relative to the total weight of the aqueous composition.

Specifically, it has been found that, in this particular embodiment, all the more so when the aqueous composition is deposited after the cosmetic composition in accordance with the invention, the presence of this film-forming polymer makes it possible to limit the water evaporation and thus to improve the durability of the desired volumizing effect.

According to another preferred embodiment, the aqueous composition contains, as film-forming polymer, a polymer dispersed in an aqueous phase, or latex. Mention may be made especially of acrylate copolymers as a 40% emulsion, such as those sold by the company Interpolymer under the name Syntran 5760®.

Advantageously, the makeup and/or care kit then comprises a cosmetic composition with an oily continuous phase also comprising, as film-forming polymer, a polymer dispersed in an aqueous phase, or latex.

Other additives may be included in the aqueous composition, as will be detailed hereinbelow: water-soluble gelling agents, dyestuffs and fillers. A few of the preferential embodiments are cited below.

Thus, according to one preferred embodiment, the aqueous composition contains a water-soluble gelling agent, as detailed below, such as a cellulose-based aqueous gel, for example based on hydroxyethylcellulose.

According to another preferred embodiment of the invention, the aqueous composition may contain fillers. As will become apparent hereinbelow, the aqueous composition is preferably transparent or translucent. For this reason, if the aqueous composition contains fillers, these fillers are themselves advantageously transparent or translucent, such that the aqueous composition preserves its transparent or translucent nature.

According to one aspect of the present invention, kits in which the aqueous composition is transparent or translucent are preferred.

The term “transparent or translucent” means the capacity to let light pass through without deviating it by means of refraction or reflection.

More particularly, the term “transparency or translucency” means the capacity of a thickness of 10 microns of the aqueous composition to transmit on average at least 25% and preferably 50% of the light in a wavelength range of 400-700 nanometres.

Surfactant

According to one preferred embodiment, the cosmetic composition in accordance with the invention may contain at least one surfactant. In accordance with a preferred aspect of this particular embodiment, the surfactant may advantageously have an HLB of greater than or equal to 6 at 25° C.

According to one particular embodiment, the surfactant(s) is (are) present in a weight content of greater than or equal to 0.1% relative to the total weight of the cosmetic composition in accordance with the invention. It (they) may especially be present in a proportion ranging from 0.1% to 30%, better still from 0.5% to 15% and better still from 1.5% to 10% by weight relative to the total weight of the cosmetic composition.

It is understood that when the polyelectrolyte is incorporated into the composition in accordance with the invention in the form of a composition already formulated with a surfactant, the amount of surfactant defined above takes into account the amount of the said surfactant included in the polyelectrolyte formulation.

The term “HLB of greater than or equal to 6” means a surfactant having at 25° C. an HLB (hydrophilic-lipophilic balance), within the Griffin meaning, of greater than or equal to 6.

The HLB value according to Griffin is defined in J. Soc. Cosm. Chem. 1954 (volume 5), pp 249-256.

Reference may be made to the document “Encyclopedia of Chemical Technology, Kirk-Othmer”, volume 22, pp 333-432, 3rd edition, 1979, Wiley, for the definition of the properties and functions (emulsifying) of surfactants, in particular pp 347-377 of this reference, for the nonionic surfactants.

The surfactant that may be included in the cosmetic composition included in the kit according to the present invention may be ionic, nonionic or of mixed ionic and nonionic nature.

Among the nonionic surfactants that may be present in the composition with an oily continuous phase, used alone or as a mixture, mention may be made especially of:

oxyethylenated and/or oxypropylenated ethers (which may comprise from 1 to 150 oxyethylene and/or oxypropylene groups) of glycerol;

oxyethylenated and/or oxypropylenated ethers (which may comprise from 1 to 150 oxyethylene and/or oxypropylene groups) of fatty alcohols (especially of C₈-C₂₄ and preferably C₁₂-C₁₈ alcohol), such as oxyethylenated cetearyl alcohol ether containing 30 oxyethylene groups (CTFA name “Ceteareth-30”) and the oxyethylenated ether of the mixture of C₁₂-C₁₅ fatty alcohols comprising 7 oxyethylene groups (CTFA name “C₁₂₋₁₅ Pareth-7” sold under the name Neodol 25-7® by Shell Chemicals);

fatty acid esters (especially of a C₈-C₂₄ and preferably C₁₆-C₂₂ acid) of polyethylene glycol (which may comprise from 1 to 150 ethylene glycol units), such as PEG-50 stearate and PEG-40 monostearate sold under the name Myrj 52P® by the company ICI Uniqema;

fatty acid esters (especially of a C₈-C₂₄ and preferably C₁₆-C₂₂ acid) of oxyethylenated and/or oxypropylenated glyceryl ethers (which may comprise from 1 to 150 oxyethylene and/or oxypropylene groups), for instance PEG-200 glyceryl monostearate sold, for example, under the name Simulsol 220 TM® by the company SEPPIC; glyceryl stearate polyethoxylated with 30 ethylene oxide groups, for instance the product Tagat S® sold by the company Goldschmidt, glyceryl oleate polyethoxylated with 30 ethylene oxide groups, for instance the product Tagat O® sold by the company Goldschmidt, glyceryl cocoate polyethoxylated with 30 ethylene oxide groups, for instance the product Varionic LI 13® sold by the company Sherex, glyceryl isostearate polyethoxylated with 30 ethylene oxide groups, for instance the product Tagat L® sold by the company Goldschmidt, and glyceryl laurate polyethoxylated with 30 ethylene oxide groups, for instance the product Tagat I® from the company Goldschmidt;

fatty acid esters (especially of a C₈-C₂₄ and preferably C₁₆-C₂₂ acid) of oxyethylenated and/or oxypropylenated sorbitol ethers (which may comprise from 1 to 150 oxyethylene and/or oxypropylene groups), for instance polysorbate 60 sold, for example, under the name Tween 60® by the company Uniqema, and also polysorbate 80, polysorbate 40 and polysorbate 20;

dimethicone copolyol, such as the product sold under the name Q2-5220® by the company Dow Corning;

dimethicone copolyol benzoate (Finsolv SLB 101® and 201® by the company Finetex);

copolymers of propylene oxide and of ethylene oxide, also known as EO/PO polycondensates;

and mixtures thereof.

The EO/PO polycondensates are more particularly copolymers consisting of polyethylene glycol and polypropylene glycol blocks, for instance polyethylene glycol/polypropylene glycol/polyethylene glycol triblock polycondensates. These triblock polycondensates have, for example, the following chemical structure:
H—(O—CH₂—CH₂)ₐ—(O—CH(CH₃)—CH₂)_{b}—(O—CH₂—CH₂)ₐ—OH,
in which formula a ranges from 2 to 120 and b ranges from 1 to 100.

The EO/PO polycondensate preferably has a weight-average molecular weight ranging from 1000 to 15 000 and better still ranging from 2000 to 13 000. Advantageously, the said EO/PO polycondensate has a cloud point, at 10 g/l in distilled water, of greater than or equal to 20° C. and preferably greater than or equal to 60° C. The cloud point is measured according to ISO standard 1065. As EO/PO polycondensates that may be used according to the invention, mention may be made of the polyethylene glycol/polypropylene glycol/polyethylene glycol triblock polycondensates sold under the name Synperonic®, for instance Synperonic PE/L44® and Synperonic PE/F127®, by the company ICI.

Among the ionic surfactants, which may be anionic or cationic, which may be present in the composition with an oily continuous phase, used alone or as a mixture, mention may be made especially of:

silicone surfactants, for instance dimethicone copolyol phosphates such as the product sold under the name Pecosil PS 100® by the company Phoenix Chemical,

amino acid derivatives, such as lauryl sarcosinate and lauryl taurate,

C₁₆-C₃₀ fatty acid salts, especially those derived from amines, for instance triethanolamine stearate;

polyoxyethylenated fatty acid salts, especially those derived from amines or alkali metal salts, and mixtures thereof;

phosphoric esters and salts thereof, such as “DEA oleth-10 phosphate” (Crodafos N 10N® from the company Croda);

sulfosuccinates such as “Disodium PEG-5 citrate lauryl sulfosuccinate” and “Disodium ricinoleamido MEA sulfosuccinate”;

alkyl ether sulfates, such as sodium lauryl ether sulfate;

isethionates;

acylglutamates such as “Disodium hydrogenated tallow glutamate” (Amisoft HS-21 R® sold by the company Ajinomoto), and mixtures thereof.

Triethanolamine stearate is most particularly suitable for the invention. This surfactant is generally obtained by simple mixing of stearic acid and triethanolamine.

Representative cationic surfactants that may especially be mentioned include:

alkylimidazolidiniums, such as isostearylethylimidonium ethosulfate,

ammonium salts, such as N,N,N-trimethyl-1-docosanaminium chloride (behentrimonium chloride).

The composition with an oily continuous phase may also contain one or more amphoteric surfactants, for instance N-acylamino acids such as N-alkylaminoacetates and cocoamphodiacetate.

It will obviously be arranged for the surfactant to be chosen and to be present in an amount such that the cosmetic composition in accordance with the invention is indeed in the form of an emulsion with an oily continuous phase.

Moreover, the cosmetic composition in accordance with the present invention preferably comprises one or more surfactant(s) with an HLB of greater than or equal to 8 and even more preferably with an HLB of greater than or equal to 10 or better still greater than or equal to 12 at 25° C.

Since it is a composition with an oily continuous phase, the cosmetic composition in accordance with the invention may also contain a surfactant with a low HLB, i.e. less than 6, among which mention may be made of:

a) nonionic surfactants with an HLB of less than 6 at 25° C., such as:

saccharide esters and ethers, such as sucrose stearate, sucrose cocoate and sorbitan stearate, and mixtures thereof, for instance Arlatone 2121® sold by the company ICI;

fatty acid esters (especially of a C₈-C₂₄ and preferably C₁₆-C₂₂ acid) of polyol, especially of glycerol or of sorbitol, such as glyceryl stearate, glyceryl stearate such as the product sold under the name Tegin M® by the company Goldschmidt, glyceryl laurate such as the product sold under the name Imwitor 312® by the company Hüls, polyglyceryl-2 stearate, sorbitan tristearate or glyceryl ricinoleate;

the mixture of cyclomethicone/dimethicone copolyol sold under the name Q2-3225C® by the company Dow Corning,

fatty alcohols such as cetyl alcohol or stearyl alcohol,

ethoxylated fatty alcohols with an ethoxylation number such that the HLB is less than 6.

Surfactants with an HLB of greater than or equal to 6 at 25° C. that are most particularly suitable for use in the invention are oxyethylenated and/or oxypropylenated (possibly comprising from 1 to 150 oxyethylene groups) fatty acid esters and ethers of sorbitol, for instance polysorbate 20 with an HLB of 16.7, polysorbate 40 with an HLB of 15.6, polysorbate 60 with an HLB of 14.9 and polysorbate 80 with an HLB of 15.0.

The cosmetic composition in accordance with the invention may comprise other surfactant(s), which are introduced, for example, into the composition by introduction of the aqueous dispersion of particles of a polymer, these surfactants being those conventionally used to stabilize them.

Structuring Agent

The cosmetic composition included in the makeup kit according to the invention may comprise at least one agent for structuring the oily phase or organic solvent, chosen from waxes, semi-crystalline polymers and lipophilic gelling agents, and mixtures thereof.

The structuring agent may represent from 0.1% to 80% by weight, preferably from 0.5% to 50% and even more preferably from 1% to 40% by weight relative to the total weight of the composition.

The amount of oily structuring agent may be adjusted by a person skilled in the art as a function of the structuring properties of the said agents.

Wax(es)

The wax under consideration in the context of the present invention is generally a lipophilic compound that is solid at room temperature (25° C.), with a solid/liquid reversible change of state, having a melting point of greater than or equal to 30° C., which may be up to 200° C. and in particular up to 120° C.

By bringing the wax to the liquid form (melting), it is possible to make it miscible with oils and to form a microscopically uniform mixture, but on cooling the mixture to room temperature, recrystallization of the wax in the oils of the mixture is obtained.

In particular, the waxes that are suitable for the invention may have a melting point of greater than or equal to 45° C. and in particular greater than or equal to 55° C.

For the purposes of the invention, the melting point corresponds to the temperature of the most endothermic peak observed by thermal analysis (DSC) as described in ISO standard 11357-3; 1999. The melting point of the wax may be measured using a differential scanning calorimeter (DSC), for example the calorimeter sold under the name MDSC 2920 by the company TA Instruments.

The measuring protocol is as follows:

A sample of 5 mg of wax placed in a crucible is subjected to a first temperature rise ranging from −20° C. to 100° C., at a heating rate of 10° C./minute, it is then cooled from 100° C. to −20° C. at a cooling rate of 10° C./minute and is finally subjected to a second temperature increase ranging from −20° C. to 100° C. at a heating rate of 5° C./minute. During the second temperature increase, the variation of the difference in power absorbed by the empty crucible and by the crucible containing the sample of wax is measured as a function of the temperature. The melting point of the compound is the temperature value corresponding to the top of the peak of the curve representing the variation in the difference in absorbed power as a function of the temperature.

The waxes that may be used in the cosmetic compositions included in the makeup kit according to the invention are chosen from waxes that are solid at room temperature, of animal, plant, mineral or synthetic origin, and mixtures thereof.

The waxes that may be used in the cosmetic compositions generally have a hardness ranging from 0.01 MPa to 15 MPa, especially greater than 0.05 MPa and in particular greater than 0.1 MPa.

The hardness is determined by measuring the compression force, measured at 20° C. using the texturometer sold under the name TA-XT2 by the company Rheo, equipped with a stainless-steel cylindrical spindle 2 mm in diameter, travelling at a measuring speed of 0.1 mm/second, and penetrating the wax to a penetration depth of 0.3 mm.

The measuring protocol is as follows:

The wax is melted at a temperature equal to the melting point of the wax+10° C. The molten wax is poured into a container 25 mm in diameter and 20 mm deep. The wax is recrystallized at room temperature (25° C.) for 24 hours such that the surface of the wax is flat and smooth, and the wax is then stored for at least 1 hour at 20° C. before measuring the hardness or the tack.

The texturometer spindle is displaced at a speed of 0.1 mm/s, then penetrates the wax to a penetration depth of 0.3 mm. When the spindle has penetrated the wax to a depth of 0.3 mm, the spindle is held still for 1 second (corresponding to the relaxation time) and is then withdrawn at a speed of 0.5 mm/s.

The hardness value is the maximum compression force measured divided by the area of the texturometer cylinder in contact with the wax.

As illustrations of waxes that are suitable for the invention, mention may be made especially of hydrocarbon-based waxes, for instance beeswax, lanolin wax and Chinese insect waxes; rice bran wax, carnauba wax, candelilla wax, ouricury wax, alfalfa wax, berry wax, shellac wax, Japan wax and sumach wax; montan wax, orange wax, lemon wax, microcrystalline waxes, paraffins and ozokerite; polyethylene waxes, the waxes obtained by Fischer-Tropsch synthesis and waxy copolymers, and also esters thereof.

Mention may also be made of waxes obtained by catalytic hydrogenation of animal or plant oils containing linear or branched C₈-C₃₂ fatty chains. Among these waxes that may especially be mentioned are isomerized jojoba oil such as the trans-isomerized partially hydrogenated jojoba oil manufactured or sold by the company Desert Whale under the commercial reference Iso-Jojoba-50®, hydrogenated sunflower oil, hydrogenated castor oil, hydrogenated coconut oil, hydrogenated lanolin oil and bis(1,1,1-trimethylol-propane) tetrastearate sold under the name Hest 2T-4S® by the company Heterene.

Mention may also be made of silicone waxes and fluoro waxes.

The waxes obtained by hydrogenation of castor oil esterified with cetyl alcohol, sold under the names Phytowax ricin 16L64® and 22L73® by the company Sophim, may also be used. Such waxes are described in patent application FR-A-2 792 190.

According to one particular embodiment, the cosmetic compositions included in the makeup kit according to the invention may comprise at least one “tacky” wax, i.e. a wax with a tack of greater than or equal to 0.1 N.s and a hardness of less than or equal to 3.5 MPa.

The tacky wax used may especially have a tack ranging from 0.1 N.s to 10 N.s, in particular ranging from 0.1 N.s to 5 N.s, preferably ranging from 0.2 N.s to 5 N.s and better still ranging from 0.3 N.s to 2 N.s.

The tack of the wax is determined by measuring the change in the force (compression force) as a function of time, at 20° C., according to the protocol indicated above for the hardness.

During the 1-second relaxation time, the force (compression force) decreases greatly until it becomes zero, and then, during the withdrawal of the spindle, the force (stretching force) becomes negative and then rises again to the value 0. The tack corresponds to the integral of the curve of the force as a function of time for the part of the curve corresponding to negative values of the force. The tack value is expressed in N.s.

The tacky wax that may be used generally has a hardness of less than or equal to 3.5 MPa, in particular ranging from 0.01 MPa to 3.5 MPa, especially ranging from 0.05 MPa to 3 MPa.

Tacky waxes that may be used include a C₂₀-C₄₀ alkyl (hydroxystearyloxy)-stearate (the alkyl group containing from 20 to 40 carbon atoms), alone or as a mixture.

Such a wax is especially sold under the names Kester Wax K 82 P®, and Kester Wax K 80 P® by the company Koster Keunen.

The waxes mentioned above generally have a starting melting point of less than 45° C.

In the present invention, waxes provided in the form of small particles having a dimension expressed as the mean “effective” volume diameter D[4.3] of about from 0.5 to 30 micrometres, in particular from 1 to 20 micrometres and more particularly from 5 to 10 micrometres, which are referred to hereinafter as “microwaxes”, may also be used. For the purpose of distinction, the waxes used according to the invention in the form of fragments of larger size are referred to hereinbelow as “conventional waxes”.

The particle sizes may be measured by various techniques; mention may be made in particular of light-scattering techniques (dynamic and static), Coulter counter methods, sedimentation rate measurements (related to the size via Stokes' law) and microscopy. These techniques make it possible to measure a particle diameter and, for some of them, a particle size distribution.

The sizes and size distributions of the particles in the cosmetic compositions are preferably measured by static light scattering using a commercial granulometer such as the MasterSizer 2000 from Malvern. The data are processed on the basis of the Mie scattering theory. This theory, which is exact for isotropic particles, makes it possible to determine an “effective” particle diameter in the case of non-spherical particles. This theory is described especially in the publication by Van de Hulst, H. C., “Light Scattering by Small Particles,” Chapters 9 and 10, Wiley, New York, 1957.

The cosmetic composition is characterized by its mean “effective” diameter by volume D[4.3], defined in the following manner:

$$D [ 4.3 ] = \frac{{\sum }_{i} {V}_{i} \cdot {d}_{i}}{{\sum }_{i} {V}_{i}}$$

in which Vᵢ represents the volume of the particles with an effective diameter dᵢ. This parameter is described especially in the technical documentation of the granulometer.

The measurements are performed at 25° C. on a dilute particle dispersion, obtained from the cosmetic composition in the following manner: 1) dilution by a factor of 100 with water, 2) homogenization of the solution, 3) standing of the solution for 18 hours, 4) recovery of the whitish uniform supernatant.

The “effective” diameter is obtained by taking a refractive index of 1.33 for water and a mean refractive index of 1.42 for the particles.

As microwaxes that may be used in the cosmetic compositions in accordance with the invention, mention may be made of carnauba microwaxes, such as the product sold under the name MicroCare 350® by the company Micro Powders, synthetic microwaves, such as the product sold under the name MicroEase 114S® by the company Micro Powders, microwaxes consisting of a mixture of carnauba wax and polyethylene wax, such as the products sold under the names Micro Care 300® and 310® by the company Micro Powders, microwaxes consisting of a mixture of carnauba wax and of synthetic wax, such as the product sold under the name Micro Care 325® by the company Micro Powders, polyethylene microwaxes, such as the products sold under the names Micropoly 200®, 220®, 220L® and 250S® by the company Micro Powders, and polytetrafluoroethylene micropowders such as the products sold under the names Microslip 519® and 519 L® by the company Micro Powders.

In the cosmetic composition included in the makeup kit according to the invention, it is obviously possible to use a mixture of waxes and especially to use one or more conventional waxes, such as, especially, a tacky wax and one or more waxes known as microwaves. The said cosmetic composition may comprise a content of waxes ranging from 0.1% to 70% by weight relative to the total weight of the cosmetic composition; it may in particular contain from 0.5% to 50% and more particularly from 1% to 30% thereof.

Semi-Crystalline Polymers

The term “polymer” means compounds containing at least two repeating units, preferably at least three repeating units and more especially at least ten repeating units. The term “semi-crystalline polymer” means polymers comprising a crystallizable portion, a crystallizable side chain or a crystallizable block in the skeleton, and an amorphous portion in the skeleton and having a first-order reversible phase-change temperature, in particular of melting (solid-liquid transition). When the crystallizable portion is in the form of a crystallizable block of the polymer skeleton, the amorphous portion of the polymer is in the form of an amorphous block; in this case, the semi-crystalline polymer is a block copolymer, for example, of the diblock, triblock or multiblock type, comprising at least one crystallizable block and at least one amorphous block. The term “block” generally means at least five identical repeating units. The crystallizable block(s) is (are) of chemical nature different than that of the amorphous block(s).

The semi-crystalline polymer has a melting point of greater than or equal to 30° C. (especially ranging from 30° C. to 80° C.), preferably ranging from 30° C. to 60° C. This melting point is a first-order change of state temperature.

This melting point may be measured by any known method and in particular using a differential scanning calorimeter (DSC).

Advantageously, the semi-crystalline polymer(s) to which the invention applies have a number-average molecular mass of greater than or equal to 1000. Advantageously, the semi-crystalline polymer(s) of the cosmetic composition in accordance with the invention have a number-average molecular mass Mn ranging from 2000 to 800 000, preferably from 3000 to 500 000, better still from 4000 to 150 000, especially less than 100 000 and better still from 4000 to 99 000. Preferably, they have a number-average molecular mass of greater than 5600, for example ranging from 5700 to 99 000. For the purposes of the invention, the term “crystallizable chain or block” means a chain or block which, if it were alone, would reversibly change from the amorphous state to the crystalline state, depending on whether the system is above or below the melting point. For the purposes of the invention, a chain is a group of atoms, which is pendent or lateral relative to the polymer skeleton. A block is a group of atoms belonging to the skeleton, this group constituting one of the repeating units of the polymer. Advantageously, the “crystallizable side chain” may be a chain containing at least six carbon atoms.

The semi-crystalline polymer may be chosen from block copolymers comprising at least one crystallizable block and at least one amorphous block, and homopolymers and copolymers bearing at least one crystallizable side chain per repeating unit, and mixtures thereof.

Such polymers are described, for example, in document EP 1 396 259.

A. Semi-Crystalline Polymers Containing Crystallizable Side Chains

Mention may be made in particular of those defined in documents U.S. Pat. No. 5,156,911 and WO-A-01/19333. They are homopolymers or copolymers comprising from 50% to 100% by weight of units resulting from the polymerization of one or more monomers bearing a crystallizable hydrophobic side chain.

These homopolymers or copolymers are of any nature, provided that they meet the conditions mentioned previously.

B. Polymers Bearing in the Skeleton at Least One Crystallizable Block

These polymers are especially block copolymers consisting of at least two blocks of different chemical nature, one of which is crystallizable.

The block polymers defined in patent U.S. Pat. No. 5,156,911 may be used;

The block copolymers of olefin or of cycloolefin containing a crystallizable chain, for instance those derived from the block polymerization of:

cyclobutene, cyclohexene, cyclooctene, norbornene (i.e. bicyclo(2,2,1)-2-heptene), 5-methylnorbornene, 5-ethylnorbornene, 5,6-dimethylnorbornene, 5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopenta-diene, or mixtures thereof,

with ethylene, propylene, 1-butene, 3-methyl-1-butene, 1-hexene, 4-methyl-1-pentene, 1-octene, 1-decene or 1-eicosene, or mixtures thereof,

and in particular copoly(ethylene/norbornene) blocks and (ethylene/propylene/ethylidene-norbornene) block terpolymers. Those resulting from the block copolymerization of at least 2 C₂-C₁₆, better still C₂-C₁₂ and even better still C₄-C₁₂ α-olefins such as those mentioned above and in particular block bipolymers of ethylene and of 1-octene may also be used.

The copolymers may be copolymers containing at least one crystallizable block, the rest of the copolymer being amorphous (at room temperature). These copolymers may also contain two crystallizable blocks of different chemical nature. The preferred copolymers are those that simultaneously contain at room temperature a crystallizable block and an amorphous block that are both hydrophobic and lipophilic, sequentially distributed; mention may be made, for example, of polymers containing one of the crystallizable blocks and one of the amorphous blocks below:

Block that is crystallizable by nature: a) polyester type, for instance poly(alkylene terephthalate), b) of polyolefin type, for instance polyethylenes or polypropylenes.

Amorphous and lipophilic block, for instance: amorphous polyolefins or copoly(olefin)s such as poly(isobutylene), hydrogenated polybutadiene or hydrogenated poly(isoprene).

As examples of such copolymers containing a crystallizable block and an amorphous block, mention may be made of:

α) poly(ε-caprolactone)-b-poly(butadiene) block copolymers, preferably used hydrogenated, such as those described in the article “Melting behavior of poly(ε-caprolactone)-block-polybutadiene copolymers” from S. Nojima, Macromolecules, 32, 3727-3734 (1999),

β) the hydrogenated block or multiblock poly(butylene terephthalate)-b-poly(isoprene) block copolymers cited in the article “Study of morphological and mechanical properties of PP/PBT” by B. Boutevin et al., Polymer Bulletin, 34, 117-123 (1995),

γ) the poly(ethylene)-b-copoly(ethylene/propylene) block copolymers cited in the articles “Morphology of semi-crystalline block copolymers of ethylene-(ethylene-alt-propylene)” by P. Rangarajan et al., Macromolecules, 26, 4640-4645 (1993) and “Polymer aggregates with crystalline cores: the system poly(ethylene)-poly(ethylene-propylene)” by P. Richter et al., Macromolecules, 30, 1053-1068 (1997),

δ) the poly(ethylene)-b-poly(ethylethylene) block copolymers cited in the general article “Crystallization in block copolymers” by I. W. Hamley, Advances in Polymer Science, Vol. 148, 113-137 (1999).

Preferably, the semi-crystalline polymers in the cosmetic composition in accordance with the invention are non-crosslinked.

According to one particular embodiment of the invention, the polymer is chosen from copolymers resulting from the polymerization of at least one monomer containing a crystallizable chain chosen from saturated C₁₄-C₂₄ alkyl (meth)acrylates, C₁₁-C₁₅ perfluoroalkyl (meth)acrylates, C₁₄ to C₂₄ N-alkyl(meth)acrylamides with or without a fluorine atom, vinyl esters containing C₁₄ to C₂₄ alkyl or perfluoroalkyl chains, vinyl ethers containing C₁₄ to C₂₄ alkyl or perfluoralkyl chains, C₁₄ to C₂₄ α-olefins, para-alkylstyrenes with an alkyl group containing from 12 to 24 carbon atoms, with at least one optionally fluorinated C₁ to C₁₀ monocarboxylic acid ester or amide, which may be represented by the following formula:

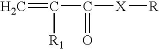

in which R₁ is H or CH₃, R represents an optionally fluorinated C₁-C₁₀ alkyl group and X represents O, NH or NR₂, in which R₂ represents an optionally fluorinated C₁-C₁₀ alkyl group.

According to a more particular embodiment of the invention, the polymer is derived from a monomer containing a crystallizable chain chosen from saturated C₁₄-C₂₂ alkyl(meth)acrylates.

As a particular example of a semi-crystalline polymer that may be used in the cosmetic composition, mention may be made of the Intelimer® products from the company Landec described in the brochure “Intelimer® Polymers”, Landec IP22 (Rev. 4-97). These polymers are in solid form at room temperature (25° C.). They bear crystallizable side chains and have the above formula X.

Lipophilic Gelling Agents

The gelling agents that may be used in the cosmetic compositions included in the makeup kits according to the invention may be organic or mineral, polymeric or molecular lipophilic gelling agents.

Mineral lipophilic gelling agents that may be mentioned include optionally modified clays, for instance hectorites modified with a C₁₀ to C₂₂ fatty acid ammonium chloride, for instance hectorite modified with distearyldimethylammonium chloride, for instance the product sold under the name Bentone 38V® by the company Elementis.

Mention may also be made of fumed silica optionally subjected to a hydrophobic surface treatment, the particle size of which is less than 1 μm. Specifically, it is possible to chemically modify the surface of the silica, by chemical reaction generating a reduced number of silanol groups present at the surface of the silica. It is especially possible to substitute silanol groups with hydrophobic groups: a hydrophobic silica is then obtained. The hydrophobic groups may be:

trimethylsiloxyl groups, which are obtained especially by treating fumed silica in the presence of hexamethyldisilazane. Silicas thus treated are known as “silica silylate” according to the CTFA (6th edition, 1995). They are sold, for example, under the references Aerosil R812® by the company Degussa, and Cab-O-Sil TS-530® by the company Cabot;

dimethylsilyloxyl or polydimethylsiloxane groups, which are obtained especially by treating fumed silica in the presence of polydimethylsiloxane or dimethyl-dichlorosilane. Silicas thus treated are known as “silica dimethyl silylate” according to the CTFA (6th edition, 1995). They are sold, for example, under the references Aerosil R972® and Aerosil R974® by the company Degussa, and Cab-O-Sil TS-610® and Cab-O-Sil TS-720® by the company Cabot.

The hydrophobic fumed silica preferably has a particle size that may be nanometric to micrometric, for example ranging from about 5 to 200 nm.

The polymeric organic lipophilic gelling agents are, for example, partially or totally crosslinked elastomeric organopolysiloxanes of three-dimensional structure, for instance those sold under the names KSG6®, KSG16® and KSG18® from Shin-Etsu, Trefil E-505C® or Trefil E-506C® from Dow Corning, Gransil SR-CYC®, SR DMF 10®, SR-DC556®, SR 5CYC gel® , SR DMF 10 gel® and SR DC 556 gel® from Grant Industries and SF 1204® and JK 113® from General Electric; ethylcellulose, for instance the product sold under the name Ethocel by Dow Chemical; polycondensates of polyamide type resulting from condensation between (α) at least one acid chosen from dicarboxylic acids containing at least 32 carbon atoms, such as fatty acid dimers, and (β) an alkylenediamine and in particular ethylenediamine, in which the polyamide polymer comprises at least one carboxylic acid end group esterified or amidated with at least one saturated and linear monoalcohol or one saturated and linear monoamine containing from 12 to 30 carbon atoms, and in particular ethylenediamine/stearyl dilinoleate copolymers such as the product sold under the name Uniclear 100 VG® by the company Arizona Chemical; galactomannans comprising from one to six and in particular from two to four hydroxyl groups per saccharide, substituted with a saturated or unsaturated alkyl chain, for instance guar gum alkylated with C₁ to C₆, and in particular C₁ to C₃, alkyl chains, and mixtures thereof Block copolymers of “diblock”, “triblock” or “radial” type, of the polystyrene/polyisoprene or polystyrene/polybutadiene type, such as the products sold under the name Luvitol HSB® by the company BASF, of the polystyrene/copoly(ethylene-propylene) type, such as the products sold under the name Kraton® by the company Shell Chemical Co., or of the polystyrene/copoly(ethylene-butylene) type, and mixtures of triblock and radial (star) copolymers in isododecane, such as those sold by the company Penreco under the name Versagel®, for instance the mixture of butylene/ethylene/styrene triblock copolymer and of ethylene/propylene/styrene star copolymer in isododecane (Versagel M 5960).

Among the lipophilic gelling agents that may be used in the cosmetic compositions included in the makeup kits according to the invention, mention may also be made of fatty acid esters of dextrin, such as dextrin palmitates, especially the products sold under the name Rheopearl TL® or Rheopearl KL® by the company Chiba Flour.

Film-Forming Polymer

According to one particular embodiment, the cosmetic composition with an oily continuous phase and the aqueous composition, included in the makeup kit according to the invention, may comprise at least one film-forming polymer.

The film-forming polymer may be present in the cosmetic composition with an oily continuous phase in a solids content ranging from 0.1% to 60% by weight, preferably from 0.5% to 40% by weight and better still from 10% to 30% by weight relative to the total weight of the composition.

The contents of film-forming polymers intrinsic to the aqueous composition have already been indicated above in the “aqueous composition” paragraph.

In the present invention, the expression “film-forming polymer” means a polymer that is capable, by itself or in the presence of an auxiliary film-forming agent, of forming a macroscopically continuous film that adheres to the keratin fibres, preferably a cohesive film and better still a film whose cohesion and mechanical properties are such that the said film can be isolated and manipulated separately, for example when the said film is made by casting on a non-stick surface, for instance a Teflon-coated or silicone-coated surface.

Among the film-forming polymers that may be used in the cosmetic composition with an oily continuous phase and the aqueous composition, included in the kit according to the invention, mention may be made of synthetic polymers, of free-radical type or of polycondensate type, and polymers of natural origin, and mixtures thereof.

These film-forming polymers are preferably different from the polyelectrolyte defined above.

The liposoluble polymers and the polymers dispersed in non-aqueous phase are preferably included in the composition with an oily continuous phase, whereas the liposoluble polymers and the polymers in dispersed form in an aqueous phase may be included both in the composition with an oily continuous phase and in the aqueous composition.

Liposoluble Polymers

According to one implementation variant, the cosmetic composition included in the makeup kit according to the invention comprises a film-forming polymer that may be a polymer dissolved in the oily phase comprising oils or organic solvents such as those described above (in which case the film-forming polymer is referred to as a liposoluble polymer).

Examples of liposoluble polymers that may be mentioned are copolymers of vinyl ester (the vinyl group being directly linked to the oxygen atom of the ester group and the vinyl ester containing a saturated, linear or branched hydrocarbon-based radical of 1 to 19 carbon atoms, linked to the carbonyl of the ester group) and of at least one other monomer, which may be a vinyl ester (other than the vinyl ester already present), an α-olefin (containing from 8 to 28 carbon atoms), an alkyl vinyl ether (in which the alkyl group comprises from 2 to 18 carbon atoms) or an allylic or methallylic ester (containing a saturated, linear or branched hydrocarbon-based radical of 1 to 19 carbon atoms, linked to the carbonyl of the ester group).

These copolymers may be crosslinked with the aid of crosslinking agents, which may be either of the vinyl type or of the allylic or methallylic type, such as tetraallyloxyethane, divinylbenzene, divinyl octanedioate, divinyl dodecanedioate and divinyl octadecanedioate.

Examples of these copolymers that may be mentioned are the following copolymers: vinyl acetate/allyl stearate, vinyl acetate/vinyl laurate, vinyl acetate/vinyl stearate, vinyl acetate/octadecene, vinyl acetate/octadecyl vinyl ether, vinyl propionate/allyl laurate, vinyl propionate/vinyl laurate, vinyl stearate/1-octadecene, vinyl acetate/1-dodecene, vinyl stearate/ethyl vinyl ether, vinyl propionate/cetyl vinyl ether, vinyl stearate/allyl acetate, vinyl 2,2-dimethyloctanoate/vinyl laurate, allyl 2,2-dimethylpentanoate/vinyl laurate, vinyl dimethylpropionate/vinyl stearate, allyl dimethylpropionate/vinyl stearate, vinyl propionate/vinyl stearate, crosslinked with 0.2% divinylbenzene, vinyl dimethylpropionate/vinyl laurate, crosslinked with 0.2% divinylbenzene, vinyl acetate/octadecyl vinyl ether, crosslinked with 0.2% tetraallyloxyethane, vinyl acetate/allyl stearate, crosslinked with 0.2% divinylbenzene, vinyl acetate/1-octadecene, crosslinked with 0.2% divinylbenzene, and allyl propionate/allyl stearate, crosslinked with 0.2% divinylbenzene.

Examples of liposoluble film-forming polymers that may also be mentioned are liposoluble copolymers, and in particular those resulting from the copolymerization of vinyl esters containing from 9 to 22 carbon atoms or of alkyl acrylates or methacrylates, and alkyl radicals containing from 10 to 20 carbon atoms.

Such liposoluble copolymers may be chosen from polyvinyl stearate, polyvinyl stearate crosslinked with the aid of divinylbenzene, of diallyl ether or of diallyl phthalate, polystearyl (meth)acrylate, polyvinyl laurate and polylauryl (meth)acrylate, it being possible for these poly(meth)acrylates to be crosslinked with the aid of ethylene glycol dimethacrylate or tetraethylene glycol dimethacrylate.

The liposoluble copolymers defined above are known and are described in particular in patent application FR-A-2 232 303; they may have a weight-average molecular weight ranging from 2000 to 500 000 and preferably from 4000 to 200 000.

As liposoluble film-forming polymers that may be used in the invention, mention may also be made of polyalkylenes and in particular copolymers of C₂-C₂₀ alkenes, such as polybutene, alkylcelluloses with a linear or branched, saturated or unsaturated C₁-C₈ alkyl radical, for instance ethylcellulose and propylcellulose, copolymers of vinylpyrrolidone (VP) and in particular copolymers of vinylpyrrolidone and of C₂ to C₄₀ and better still C₃ to C₂₀ alkene. As examples of VP copolymers which may be used in the invention, mention may be made of the copolymers of VP/vinyl acetate, VP/ethyl methacrylate, butylated polyvinylpyrrolidone (PVP), VP/ethyl methacrylate/methacrylic acid, VP/eicosene, VP/hexadecene, VP/triacontene, VP/styrene or VP/acrylic acid/lauryl methacrylate.

Mention may also be made of silicone resins, which are generally soluble or swellable in silicone oils, which are crosslinked polyorganosiloxane polymers. The nomenclature of silicone resins is known under the name “MDTQ”, the resin being described as a function of the various siloxane monomer units it comprises, each of the letters “MDTQ” characterizing a type of unit.

Examples of commercially available polymethylsilsesquioxane resins that may be mentioned include those sold:

by the company Wacker under the reference Resin MK, such as Belsil PMS MK;

by the company Shin-Etsu under the reference KR-220L.

Siloxysilicate resins that may be mentioned include trimethyl siloxysilicate (TMS) resins such as those sold under the reference SR 1000 by the company General Electric or under the reference TMS 803 by the company Wacker. Mention may also be made of the trimethyl siloxysilicate resins sold in a solvent such as cyclomethicone, sold under the name “KF-7312J” by the company Shin-Etsu, and “DC 749” and “DC 593” by the company Dow Corning.

Silicone polyamides of the polyorganosiloxane type may also be used, such as those described in documents U.S. Pat. No. 5,874,069, U.S. Pat. No. 5,919,441, U.S. Pat. No. 6,051,216 and U.S. Pat. No. 5,981,680.

These silicone polymers may belong to the following two families:

1) polyorganosiloxanes comprising at least two groups capable of establishing hydrogen interactions, these two groups being located in the polymer chain, and/or

2) polyorganosiloxanes comprising at least two groups capable of establishing hydrogen interactions, these two groups being located on grafts or branches.

According to one embodiment of the invention, the film-forming polymer is a film-forming linear block ethylenic polymer, which preferably comprises at least one first block and at least one second block with different glass transition temperatures (Tg), the said first and second blocks being linked together via an intermediate block comprising at least one constituent monomer of the first block and at least one constituent monomer of the second block.

Advantageously, the first and second blocks of the block polymer are mutually incompatible.

Such polymers are described, for example, in document EP 1 411 069 or WO 04/028 488.

Water-Soluble Polymers

According to another implementation variant, the cosmetic composition with an oily continuous phase and the aqueous composition included in the makeup kit according to the invention comprise at least one film-forming polymer that may be a water-soluble polymer. The polymer is then dissolved in the aqueous phase of the composition. Examples of water-soluble film-forming polymers that may be mentioned include:

proteins, for instance proteins of plant origin such as wheat proteins and soybean proteins; proteins of animal origin such as keratin, for example keratin hydrolysates and sulfonic keratins;

polymers of cellulose such as hydroxyethylcellulose, hydroxypropyl-cellulose, methylcellulose and ethylhydroxyethylcellulose;

acrylic polymers or copolymers, such as polyacrylates or polymethacrylates;

vinyl polymers, for instance polyvinylpyrrolidones, copolymers of methyl vinyl ether and of maleic anhydride, the copolymer of vinyl acetate and of crotonic acid, copolymers of vinylpyrrolidone and of vinyl acetate; copolymers of vinylpyrrolidone and of caprolactam; polyvinyl alcohol;

polymers of natural origin, which are optionally modified, such as:

gum arabics, guar gum, xanthan derivatives, karaya gum;

alginates and carrageenans;

glycoaminoglycans, hyaluronic acid and derivatives thereof;

shellac resin, sandarac gum, dammar resins, elemi gums and copal resins;

deoxyribonucleic acid;

mucopolysaccharides such as chondroitin sulfate, and mixtures thereof

Polymers of Natural Origin

The polymers of natural origin, which are optionally modified, may be chosen from shellac resin, sandarac gum, dammar resins, elemi gums, copal resins and cellulose polymers, and mixtures thereof.

Polymers in Dispersed Form

The film-forming polymer may be present in the form of particles dispersed in an aqueous phase or in a non-aqueous solvent phase, generally known as a latex or pseudolatex. The techniques for preparing these dispersions are well known to those skilled in the art.

a) Aqueous Dispersion

Aqueous dispersions of film-forming polymers that may be used include the acrylic dispersions sold under the names Neocryl XK-90®, Neocryl A-1070®, Neocryl A-1090®, Neocryl BT-62®, Neocryl A-1079® and Neocryl A-523® by the company Avecia-Neoresins, Dow Latex 432® by the company Dow Chemical, Daitosol 5000 AD® or Daitosol 5000 SJ® by the company Daito Kasey Kogyo; Syntran 5760® by the company Interpolymer, Allianz Opt® by the company Rohm & Haas or the aqueous dispersions of polyurethane sold under the names Neorez R-981® and Neorez R-974® by the company Avecia-Neoresins, Avalure UR-405®, Avalure UR-410®, Avalure UR-425®, Avalure UR-450®, Sancure 875®, Avalure UR-445® and Sancure 2060® by the company Noveon, Impranil 85® by the company Bayer and Aquamere H-1511® by the company Hydromer; the sulfopolyesters sold under the brand name Eastman AQ® by the company Eastman Chemical Products, vinyl dispersions, for instance Mexomer PAM®, aqueous dispersions of polyvinyl acetate, for instance Vinybran® from the company Nisshin Chemical, or those sold by the company Union Carbide, aqueous dispersions of terpolymer of vinylpyrrolidone, dimethylaminopropylmethacrylamide and lauryldimethyl-propylmethacrylamidoammonium chloride, such as Styleze W from ISP, aqueous dispersions of polyurethane/polyacrylic hybrid polymers, such as those sold under the references Hybridur® by the company Air Products or Duromer® from National Starch, dispersions of core/shell type: for example those sold by the company Atofina under the reference Kynar (core: fluoro—shell: acrylic) or those described in document U.S. Pat. No. 5,188,899 (core: silica—shell: silicone), and mixtures thereof.

b) Non-Aqueous Dispersion

Mention may also be made of dispersions of particles of a grafted ethylenic polymer, preferably an acrylic polymer, in a liquid oily phase, the ethylenic polymer advantageously being dispersed in the absence of additional stabilizer at the surface of the particles, as described especially in document WO 04/055 081.

The cosmetic composition in accordance with the invention may comprise a plasticizer that promotes the formation of a film with the film-forming polymer. Such a plasticizer may be chosen from any compound known to those skilled in the art as being capable of fulfilling the desired function.

In this respect, mention may be made of acrylic dispersions in isododecane, for instance Mexomer PAP® from the company Chimex.

According to one preferred embodiment of the present invention, the cosmetic composition with an oily continuous phase included in the makeup kit comprises at least 10% by weight, preferably at least 15% by weight and better still at least 20% by weight of solids of at least one film-forming polymer, relative to the total weight of the composition.

According to one advantageous embodiment of the present invention, the aqueous composition included in the makeup kit comprises at least 10% by weight and preferably at least 15% by weight of solids of an aqueous dispersion of at least one film-forming polymer, relative to the total weight of the composition.

Water and/or Water-Soluble Solvent

It should be noted that the cosmetic and aqueous compositions included in the makeup kit according to the invention may comprise, independently of each other, an aqueous phase comprising water and/or at least one water-soluble solvent. Needless to say, as regards the cosmetic composition with an oily continuous phase, this amount of aqueous phase is adjusted to preserve a margin of swelling of the aqueous polyelectrolyte reservoirs that is sufficient for the volumizing effect to become apparent in terms of a makeup effect, when the two compositions are placed in contact.

In the present invention, the term “water-soluble solvent” denotes a compound that is liquid at room temperature and water-miscible (miscibility in water of greater than 50% by weight at 25° C. and atmospheric pressure).

Among the water-soluble solvents that may be used in the said cosmetic compositions, mention may be made especially of lower monoalcohols containing from 1 to 5 carbon atoms, such as ethanol and isopropanol, glycols containing from 2 to 8 carbon atoms, such as ethylene glycol, propylene glycol, 1,3-butylene glycol and dipropylene glycol, C₃ and C₄ ketones and C₂-C₄ aldehydes.

The aqueous phase (water and/or water-soluble solvent(s)) may be introduced in unmodified form into the cosmetic composition and the aqueous composition included in the makeup kit or may be incorporated therein by means of one or more ingredients constituting the said cosmetic and aqueous compositions. Thus, water may especially be introduced into the cosmetic composition by means of introducing a latex or pseudolatex, i.e. an aqueous dispersion of polymer particles, or alternatively an inverse latex, such as the various types of Simulgel® product.

The content of water and/or of water-soluble solvent(s) in the cosmetic composition with an oily continuous phase included in the makeup kit according to the invention is preferably less than 30%, preferably less than 25% or even 20% and especially 15% by weight relative to the total weight of the cosmetic composition.

Besides the abovementioned ingredients, each of the cosmetic or aqueous compositions included in the makeup kit according to the invention may comprise other additives that are standard in cosmetics, provided that the respective amounts thereof do not harm the exhibition of the desired volumizing effect on the final makeup.

Water-Soluble Gelling Agent

The aqueous composition included in the makeup kit according to the invention may comprise a water-soluble gelling agent.

The water-soluble film-forming polymers mentioned above may also act as water-soluble gelling agent. An example that may be mentioned in this respect is hydroxyethylcellulose.

The water-soluble gelling polymer may be present in the aqueous composition in a solids content ranging from 0.01% to 60% by weight, preferably from 0.25% to 40% by weight and better still from 0.5% to 30% by weight or even from 1% to 20% by weight relative to the total weight of the aqueous composition.

Dyestuff

The cosmetic and aqueous compositions included in the makeup kits according to the invention may also, independently of each other, comprise at least one dyestuff, for instance pulverulent dyes, liposoluble dyes and water-soluble dyes.

The pulverulent dyestuffs may be chosen from pigments and nacres.

The pigments may be white or coloured, mineral and/or organic, and coated or uncoated. Among the mineral pigments that may be mentioned are titanium dioxide, optionally surface-treated, zirconium oxide, zinc oxide or cerium oxide, and also iron oxide or chromium oxide, manganese violet, ultramarine blue, chromium hydrate and ferric blue. Among the organic pigments that may be mentioned are carbon black, pigments of D&C type, and lakes based on cochineal carmine or on barium, strontium, calcium or aluminium.

The nacres may be chosen from white nacreous pigments such as mica coated with titanium or with bismuth oxychloride, coloured nacreous pigments such as titanium mica with iron oxides, titanium mica with, especially, ferric blue or chromium oxide, titanium mica with an organic pigment of the abovementioned type, and also nacreous pigments based on bismuth oxychloride.

The liposoluble dyes are, for example, Sudan red, D&C Red 17, D&C Green 6, β-carotene, soybean oil, Sudan brown, D&C Yellow 11, D&C Violet 2, D&C Orange 5, quinoline yellow and annatto.

These dyestuffs may be present in a content ranging from 0.01% to 30% by weight relative to the total weight of the said cosmetic composition or of the aqueous composition.

Fillers

The cosmetic and aqueous compositions included in the makeup kits according to the invention may, independently of each other, also comprise at least one filler.

The fillers may be chosen from those that are well known to persons skilled in the art and commonly used in cosmetic and aqueous compositions. The fillers may be mineral or organic, and lamellar or spherical. Mention may be made of talc, mica, silica, kaolin, polyamide powders, for instance the Nylon® sold under the trade name Orgasol® by the company Atochem, poly-β-alanine powders and polyethylene powders, powders of tetrafluoroethylene polymers, for instance Teflon®, lauroyllysine, starch, boron nitride, expanded polymeric hollow microspheres such as those of polyvinylidene chloride/acrylonitrile, for instance the products sold under the name Expancel® by the company Nobel Industrie, acrylic powders, such as those sold under the name Polytrap® by the company Dow Corning, polymethyl methacrylate particles and silicone resin microbeads (for example Tospearls® from Toshiba), precipitated calcium carbonate, magnesium carbonate and magnesium hydrocarbonate, hydroxyapatite, hollow silica microspheres (Silica Beads® from Maprecos), glass or ceramic microcapsules, metal soaps derived from organic carboxylic acids containing from 8 to 22 carbon atoms and in particular from 12 to 18 carbon atoms, for example zinc, magnesium or lithium stearate, zinc laurate and magnesium myristate.

It is also possible to use a compound that is capable of swelling on heating and especially heat-expandable particles such as non-expanded microspheres of copolymer of vinylidene chloride/acrylonitrile/methyl methacrylate or of acrylonitrile homopolymer copolymer, for instance those sold, respectively, under the references Expancel® 820 DU 40 and Expancel® 007WU by the company Akzo Nobel.

The fillers may represent from 0.1% to 25% and in particular from 1% to 20% by weight relative to the total weight of the cosmetic composition or of the aqueous composition.

The cosmetic and aqueous compositions included in the makeup kits according to the invention may, independently of each other, also comprise any additive usually used in cosmetics, such as antioxidants, preserving agents, fibres, fragrances, neutralizers, vitamins, coalescers and plasticizers, and mixtures thereof.

Fibres

The cosmetic and aqueous compositions included in the makeup kits according to the invention may, independently of each other, also comprise fibres that especially allow an improvement in the lengthening effect in the field of making up keratin fibres.

The term “fibre” should be understood as meaning an object of length L and diameter D such that L is very much greater than D, D being the diameter of the circle in which the cross section of the fibre is inscribed. In particular, the ratio L/D (or shape factor) is chosen in the range from 3.5 to 2500, especially from 5 to 500 and more particularly from 5 to 150.

The fibres that may be used in the compositions included in the makeup kits of the invention may be mineral or organic fibres of synthetic or natural origin. They may be short or long, individual or organized, for example braided, and hollow or solid. They may have any shape, and may especially have a circular or polygonal (square, hexagonal or octagonal) cross section, depending on the intended specific application. In particular, their ends are blunt and/or polished to prevent injury.

In particular, the fibres have a length ranging from 1 μm to 10 mm, preferably from 0.1 mm to 5 mm and better still from 0.3 mm to 3.5 mm. Their cross section may be within a circle of diameter ranging from 2 nm to 500 μm, particularly ranging from 100 nm to 100 μm and more particularly from 1 μm to 50 μm. The weight or yarn count of the fibres is often given in denier or decitex, and represents the weight in grams per 9 km of yarn. In particular, the fibres may have a yarn count chosen in the range from 0.15 to 30 denier and better still from 0.18 to 18 denier.

The fibres that may be used in the compositions included in the makeup kits according to the invention may be chosen from rigid and non-rigid fibres, and may be of synthetic or natural, mineral or organic origin.

Moreover, the fibres may or may not be surface-treated, may be coated or uncoated, and may be coloured or uncoloured.

As fibres that may be used in the compositions included in the makeup kits according to the invention, mention may be made of non-rigid fibres such as polyamide (Nylon®) fibres or rigid fibres such as polyimideamide fibres, for instance those sold under the names Kermel® and Kermel Tech® by the company Rhodia or poly(p-phenyleneterephthalamide) (or aramid) fibres sold especially under the name Kevlar® by the company DuPont de Nemours.

The fibres may be present in the cosmetic composition or the aqueous composition in a content ranging from 0.01% to 10% by weight, in particular from 0.05% to 5% by weight and more particularly from 0.1% to 3% by weight relative to the total weight of the cosmetic composition or of the aqueous composition.

Cosmetic Active Agents

The cosmetic and aqueous compositions included in the makeup kits according to the invention may, independently of each other, further comprise any ingredient conventionally used in cosmetics. These ingredients may be chosen especially from polymers, especially fixing polymers; hair-conditioning agents; opacifiers; fragrances; thickeners; gelling agents; hair dyes; silicone resins; silicone gums; preserving agents; antioxidants; cosmetic active agents; sunscreens; pH stabilizers; vitamins; moisturizers; antiperspirants; deodorants; self-tanning compounds, and mixtures thereof. The amounts of these various ingredients are those conventionally used in the fields under consideration, for example from 0.01% to 20% by weight relative to the total weight of the cosmetic composition, and from 0.1% to 10% by weight relative to the total weight of the aqueous composition.

Needless to say, a person skilled in the art will take care to select the optional additional additives and/or the amount thereof such that the advantageous properties of the said cosmetic and aqueous compositions are not, or are not substantially, adversely affected by the envisaged addition.

Formulation

The cosmetic composition included in the makeup kit according to the invention may be in liquid, pasty, solid, foam or spray form.

The kit according to the invention may be used for making up human skin, lips and/or keratin fibres. The kit thus finds a particular application as a kit for making up the body or the face, such as a foundation, a lipstick, a lipcare product, a nail varnish, a nailcare product, a mascara or an eyeliner; a haircare kit such as a hair dyeing or hairstyle hold kit, or alternatively an antisun kit.

According to one preferred aspect of the invention, the kit is in the form of lipsticks or complexion products especially such as foundations, or alternatively mascaras.

According to one particular embodiment, i.e. when the aqueous composition is applied second, the aqueous compositions included in the makeup kit according to the invention may be in any form suitable for an application of “top coat” type. As outlined above, they may advantageously be in the form of a gel.

Preparation Process

In any case, the cosmetic compositions and the aqueous compositions included in the makeup kit according to the invention may be prepared according to methods known to those skilled in the art.

In the case of a kit for making up keratin fibres, the process for preparing the compositions according to the invention depends on the desired type of mascara.

Non-Therapeutic Makeup and/or Care Process

The present invention relates to a non-therapeutic cosmetic process for making up and/or caring for the skin, the lips and/or keratin fibres, comprising at least one step of applying to the skin, the lips and/or keratin fibres a cosmetic composition as defined above, and at least one step of applying an aqueous composition.

A subject of the present invention is also a non-therapeutic process for making up and/or caring for keratin fibres, comprising at least one step of applying to the said keratin fibres, especially the eyelashes, a cosmetic composition as defined above, and at least one step of applying an aqueous composition. The two steps may advantageously be performed in the preferential order of application of the cosmetic composition followed by the step of applying the aqueous composition. In this way, the volumizing or swelling effect on the eyelashes is optimally exhibited.

The kit according to the present invention may advantageously comprise one or more means for applying the cosmetic and aqueous compositions to the surface to be made up.

The cosmetic compositions and the aqueous compositions included in the makeup kit of the invention, when they are intended for making up keratin fibres, may be applied in particular to the eyelashes using a brush or a comb.

The thickening effect of the makeup may moreover be boosted most particularly by selecting the means for successive application of the cosmetic and aqueous compositions, which may in particular be a makeup brush.

In the present case, it is particularly advantageous, in the case of making up the eyelashes, to apply at least one of the said compositions with a makeup brush as described in patents FR 2 701 198, FR 2 605 505, EP 792 603 and EP 663 161.

According to one particular embodiment, the makeup kit according to the invention may comprise at least two separate packagings, one comprising the cosmetic composition defined above and the other comprising the aqueous composition also defined above.

In reality, especially in the case of making up keratin fibres, this operation being performed by means of a multiple action by the user, i.e. in at least two steps, the first consisting in applying the “base coat” composition and the second consisting in applying the “top coat” composition totally or partially over the said cosmetic composition, a makeup kit packaged in a single packaging is particularly suitable. This alternative constitutes a preferred embodiment of the invention.

When the kit is in the form of a single packaging, it may be presented as a container delimiting at least one compartment or reservoir that comprises the “base coat” composition, the said compartment being closed by means of a closing member, and at least one compartment or reservoir that comprises the “top coat” composition, also being closed by means of a closing member.

Again, when the kit is in the form of a single packaging, this packaging is preferably combined with at least one application means or applicator, especially in the form of a brush comprising an arrangement of bristles maintained by a twisted wire. Such a twisted brush is especially described in patent U.S. Pat. No. 4,887,622. It may also be in the form of a comb comprising a plurality of application members, obtained especially by moulding. Such combs are described, for example, in patent FR 2 796 529. The applicator may be solidly attached to the container, as described, for example, in patent FR 2 761 959. Advantageously, the applicator is solidly attached to a stem, which is itself solidly attached to the closing member.

The closing member may be coupled to the container by screwing. Alternatively, the coupling between the closing member and the container takes place other than by screwing, especially via a bayonet mechanism, by click-fastening or by tightening. The term “click-fastening” in particular means any system involving the passing of a rim or bead of material by elastic deformation of a portion, especially of the closing member, followed by return to the elastically unstressed position of the said portion after the rim or bead has been passed.

The container, advantageously comprising two compartments or reservoirs, may be at least partly made of thermoplastic material. Examples of thermoplastic materials that may be mentioned include polypropylene and polyethylene.

Alternatively, the container is made of a non-thermoplastic material, especially of glass or metal (or alloy).

The container is preferably equipped with a drainer located in the region of at least one aperture of the container. Such a drainer makes it possible to wipe the applicator and, optionally, the stem to which it may be solidly attached. Such a drainer is described, for example, in patent FR 2 792 618.

The content of the patents or patent applications mentioned previously are incorporated by reference into the present patent application.

According to one particularly preferred embodiment, the makeup kit comprises two reservoirs each comprising one among the “base coat” and “top coat” compositions, each of the reservoirs being equipped with a makeup brush, especially of mascara brush type as described above.

The examples that follow are presented as non-limiting illustrations of the invention. Unless otherwise indicated, the amounts are given in grams.

The tests that follow were used to evaluate the swelling.

Specimen and Makeup Application

False eyelash specimens were made with straight black Caucasian hair with a fringe length of 19 mm. The said fringes were mounted between two 30 mm by 30 mm plates.

Description of the Test

Hair was made up in two steps:

Step 1: 30 sweeps of a mascara brush with the “base coat” cosmetic composition with an oily continuous phase,

Step 2: 20 sweeps of a mascara brush with the “top coat” aqueous composition.

The volume of the base alone before swelling (V_{b}) after step 1 was evaluated by photographic analysis, and the volume after swelling after applying the base and the top coat (V_{b+t}) after step 2 was also evaluated by photographic analysis.

### EXAMPLE 1

### Wax-Free Mascara

The results relating to the swelling and also the contents of the constituents of compositions of the cosmetic composition with an oily continuous phase type in accordance with the invention are collated in Table 1 below.

The aqueous composition, referred to as the “top coat”, is an aqueous gel containing 3.3% by weight of hydroxyethylcellulose.

TABLE 1

| | 0 | 1 | 2 | 3 | 4 |
|---|---|---|---|---|---|
| | (compar- | (inven- | (inven- | (inven- | (inven- |
| Test No. Base | ative) | tion) | tion) | tion) | tion) |
| Vinyl acetate/ | 30 | 30 | 30 | 30 | 30 |
| allyl stearate | | | | | |
| copolymer | | | | | |
| Dye | 5.7 | 5.7 | 5.7 | 5.7 | 5.7 |
| Bentone | 3.7 | 3.7 | 3.7 | 3.7 | 3.7 |
| Propylene | 1.2 | 1.2 | 1.2 | 1.2 | 1.2 |
| carbonate | | | | | |
| Isododecane | qs 100 | qs 100 | qs 100 | qs 100 | qs 100 |
| Polysorbate 80 | 0 | 0 | 2 | 0 | 2 |
| (Tween 80 V ® | | | | | |
| from Uniqema) | | | | | |
| Acrylamide/Na | 0 | 10 | 10 | 0 | 0 |
| AMPS copolymer | | | | | |
| (Simulgel 600 ® | | | | | |
| from SEPPIC) | | | | | |
| Sodium poly- | 0 | 0 | 0 | 4 | 4 |
| acrylate | | | | | |
| (Cosmedia SP | | | | | |
| from Cognis) | | | | | |
| Degree of | 1.2 | 2.0 | 5.3 | 3.3 | 3.8 |
| swelling | | | | | |
| V_{b+t}/V_{b} | | | | | |

These results clearly show the swelling afforded by the successive application of a cosmetic composition with an oily continuous phase in accordance with the invention, followed by the application of an aqueous composition to the eyelashes.

Further improved swelling is observed when the cosmetic composition with an oily continuous phase also contains a surfactant.

### EXAMPLE 2

### Kit

% by weight

a) Composition with an oily continuous phase (base coat)

| | |
|---|---|
| Beeswax | 8.0 |
| Vinyl acetate/allyl stearate copolymer (65/35) | 20.0 |
| Methacrylic acid/methyl methacrylate/butyl acrylate/ | 10.0 |
| cetyleicosenyl methacrylate tetrapolymer as an aqueous | |
| dispersion containing 48% by weight of AM, 6% | |
| propylene glycol (Allianz Opt from Rohm & Haas) | |
| Polybutene (MW 2060) | 1.0 |
| Propylene carbonate | 1.2 |
| Polysorbate 80 (Tween 80 V ® from Uniqema) | 2.0 |
| Acrylamide/Na AMPS copolymer in isohexadecane with | 10.0 |
| polysorbate 80 (Simulgel 600 ® from SEPPIC) | |
| Distearyldimethylammonium-modified hectorite | 3.7 |
| Polytetrafluoroethylene powder (Microslip 519L | 5.0 |
| from Micropowders) | |
| Dye | 5.7 |
| Preserving agents | qs |
| Isododecane | qs 100 |

b) Aqueous composition (top coat)

| | |
|---|---|
| Aliphatic polyurethane as an aqueous dispersion containing | 46.0 |
| 38% active material (Avalure UR 450 ® de Noveon) | |
| Hydroxyethylcellulose | 1.5 |
| Preserving agent | qs |
| Water | qs 100 |

A first coat of the composition with an oily continuous phase is applied to the eyelashes, followed by a second coat of the aqueous composition. Swelling of the final deposit on the eyelashes and a volumizing effect on the eyelashes are observed.

What is claimed is:

1. A kit for making up and/or caring for skin, lips and/or keratin fibers, comprising:
a cosmetic composition having an oily continuous phase comprising at least one polyelectrolyte; and
an aqueous composition.

2. The kit according to claim 1, wherein the polyelectrolyte comprises at least one member selected from the group consisting an acrylamide/2-methyl-2-[(1-oxo-2-propenyl)amino]-1-propanesulfonic acid copolymer, a crosslinked starch glycolate in powder form, a polyacrylate, a starch-based grafted copolymer, ionizable polysaccharide derivatives, polyacrylic acids, polyacrylic acid/alkyl acrylate copolymers, AMPS (polyacrylamidomethylpropanesulfonic acid partially neutralized with ammonia and highly crosslinked), and AMPS/alkyl methacrylate polyoxyethylenated copolymers (crosslinked or non-crosslinked).

3. The kit according to claim 1, wherein the polyelectrolyte comprises at least one member selected from the group consisting of an acrylamide/2-methyl-2-[(1-oxo-2-propenyl)amino]-1-propanesulfonic acid copolymer, a polyacrylate, and copolymers thereof.

4. The kit according to claim 1, wherein the polyelectrolyte is present in an amount of at least 0.05% by weight relative to a total weight of the cosmetic composition having the oily continuous phase.

5. The kit according to claim 1, wherein the polyelectrolyte is present in an amount of from 0.05% to 15% by weight relative to a total weight of the composition having the oily continuous phase.

6. The kit according to claim 1, wherein the cosmetic composition having the oily continuous phase comprises oil in an amount of from 10% to 90% by weight relative to a total weight of the composition having the oily continuous phase.

7. The kit according to claim 1, wherein one or more oils forming the oily continuous phase comprise at least one member selected from the group consisting of volatile oils and non-volatile oils.

8. The kit according to claim 7, wherein the volatile oils comprise at least one member selected from the group consisting of hydrocarbon-based oils, silicone oils and fluoro oils.

9. The kit according to claim 1, wherein the cosmetic composition having the oily continuous phase further comprises a surfactant.

10. The kit according to claim 9, wherein the surfactant has an HLB of at least 6 at 25° C.

11. The kit according to claim 9, wherein the surfactant is present in an amount of from 0.1% to 30% by weight relative to a total weight of the cosmetic composition having the oily continuous phase.

12. The kit according to claim 1, wherein the cosmetic composition having the oily continuous phase further comprises a structuring agent comprising at least one member selected from the group consisting of waxes, semi-crystalline polymers, and lipophilic gelling agents.

13. The kit according to claim 1, wherein the cosmetic composition having the oily continuous phase further comprises a film-forming polymer.

14. The kit according to claim 13, wherein the film-forming polymer comprises at least one member selected from the group consisting of a liposoluble polymer, a water-soluble polymer, a polymer of natural origin, and a polymer in dispersed form in an aqueous phase or in an oily phase.

15. The kit according to claim 1, wherein the film-forming polymer comprises a polymer in aqueous dispersion.

16. The kit according to claim 1, wherein the cosmetic composition having the oily continuous phase comprises at least one of water and at least one water-soluble solvent in an amount of less than 30% by weight relative to a total weight of the cosmetic composition having the oily continuous phase.

17. The kit according to claim 1, wherein the aqueous composition comprises at least one film-forming polymer.

18. The kit according to claim 17, wherein the film-forming polymer comprises a polymer in aqueous dispersion.

19. The kit according to claim 1, wherein the aqueous composition comprises a gelling agent.

20. The kit according to claim 19, wherein the gelling agent comprises a cellulose derivative.

21. The kit according to claim 19, wherein the gelling agent comprises hydroxyethylcellulose.

22. The kit according to claim 1, wherein the aqueous composition is transparent or translucent.

23. The kit according to claim 1, wherein at least one of the cosmetic composition having the oily continuous phase and the aqueous composition further comprises at least one member selected from the group consisting of dyestuffs, fillers, and fibers.

24. The kit according to claim 1, wherein the kit is in a form for dispensing a lipstick, a complexion product, or a mascara.

25. The kit according to claim 1, further comprising means for applying the cosmetic composition having the oily continuous phase and the aqueous composition to a surface to be made up.

26. The kit according to claim 1, further comprising a first package containing the cosmetic composition having the oily continuous phase and a second package containing the aqueous composition.

27. The kit according to claim 1, wherein components of the kit are provided in a single package.

28. The kit according to claim 27, wherein the single package comprises a first compartment containing the cosmetic composition having the oily continuous phase and a second compartment containing the aqueous composition.

29. The kit according to claim 28, wherein the kit further comprises a closing member capable of closing at least one of the first compartment and the second compartment.

30. The kit according to claim 1, wherein the kit is in a form for dispensing a mascara and comprises at least one application means.

31. The kit according to claim 30, wherein the at least one application means comprises a brush or a comb.

32. A cosmetic process for making up and/or caring for skin, lips and/or keratin fibers, comprising:
applying a cosmetic composition having an oily continuous phase to the skin, lips and/or keratin fibers, the cosmetic composition having the oily continuous phase comprising at least one polyelectrolyte; and
applying an aqueous composition to the skin, lips and/or keratin fibers.

33. The process according to claim 32, wherein the aqueous composition comprises a film-forming polymer in aqueous dispersion.

34. The process according to claim 32, wherein the cosmetic composition having the oily continuous phase is applied before the aqueous composition.

35. A made-up support, formed by:
applying a cosmetic composition having an oily continuous phase to a support, the cosmetic composition having the oily continuous phase comprising at least one polyelectrolyte; and
applying an aqueous composition to the support, the aqueous composition comprising a film-forming polymer in aqueous dispersion.

36. A method for preparing a kit for making up skin, lips and/or keratin fibers, comprising:
preparing a cosmetic composition having an oily continuous phase and an aqueous composition;
wherein preparing the cosmetic composition having the oily continuous phase comprises employing at least one polyelectrolyte as an additive for the cosmetic composition having the oily continuous phase to obtain a volumizing effect when the compositions are placed in contact.
